(19) United States
(12) Patent Application Publication
(10) Pub. No.: US 2025/0044268 A1
(43) Pub. Date: Feb. 6, 2025

(54) METHOD AND APPARATUS FOR RAPID ROCK AND SOIL TESTING

(71) Applicant: Anton Grabreck, Penticton (CA)

(72) Inventor: Anton Grabreck, Penticton (CA)

(21) Appl. No.: 18/365,833

(22) Filed: Aug. 4, 2023

(51) Int. Cl. G01N 33/00; G01N 31/00; G01N 33/24

(52) U.S. Cl. CPC G01N 33/0062; G01N 31/005; G01N 33/24

(57) ABSTRACT

An apparatus and method for testing geological samples is disclosed wherein the apparatus comprising a body sized to be located over a testing location defining a cavity therein, a reservoir containing an oxidizing agent, an applicator configured to dispense a quantity of the oxidising agent from the reservoir to a sample located with or under the cavity and a sensor adapted to measure a concentration of hydrogen sulphide within the cavity. The method comprises locating the body over a sample to be tested, measuring an initial hydrogen sulfide concentration within the cavity, introducing a quantity of an oxidizing agent to the sample, measuring a second hydrogen sulfide concentration within the cavity and comparing the initial and oxidized hydrogen sulfide concentrations to indicate a potential for the presence of a desired mineral.

### BACKGROUND

### 1. Technical Field

This disclosure relates generally to geological testing and in particular to a method and apparatus for rapid testing of geological samples.

### 2. Description of Related Art

In the fields of prospecting, it is necessary to analyze geological samples for indications that such samples may potentially contain desirable minerals or other substances. One method for conducting such analysis is to measure the amount of hydrogen sulfide being emitted from the sample to indicate potential hydrocarbon or desirable mineral presence.

One difficulty with conventional methods for measuring hydrogen sulfide emissions is that such emissions will be quite small and therefore such measurement is difficult. In particular, due to the potentially small quantity of such emissions, the relative proportion of hydrogen sulfide in a particular air sample may be quite small making detection difficult. Additionally, there may be significant potential for such sources of hydrogen sulfide to be from other than the sample in question in the field.

Accordingly, many conventional methods require obtaining samples of rock or soil and then testing in a laboratory or other controlled environments where an isolated measure of hydrogen sulfide can be obtained. Examples of such methods may be found in International Patent application publication no WO 2020/186302 to Scott, for example. It will be appreciated that such methods, as requiring the return of samples for analysis in a lab can cause a significant delay between the time of obtaining the sample any obtaining any result making prospecting of large areas time consuming.

Other previous methods have attempted to isolate an area around a rock or soil sample so as to measure the output of hydrogen sulfide therefrom. However such methods have required locating and maintaining a device over the desired location for an extended period of time to collect the gasses outputted therefrom. Disadvantageously, such methods require either a long period of time to sequentially locate the device at a plurality of locations or a large number of devices to test such plurality of locations. Examples of such devices and methods may be found at U.S. Pat. No. 4,017,731 to Howell et al.

### SUMMARY OF THE DISCLOSURE

According to a first embodiment, there is disclosed an apparatus for testing geological samples comprising a body sized to be located over a testing location defining a cavity therein, a reservoir containing an oxidizing agent, an applicator configured to dispense a quantity of the oxidising agent from the reservoir to a sample located with or under the cavity and a sensor adapted to measure a concentration of hydrogen sulphide within the cavity.

The body may be substantially frustoconical. The apparatus may further comprise a processor operable to record the initial and oxidized measurements as provided by the sensor and determine an increase in hydrogen sulfide concentration in response to the introduction of the oxidizing agent. The processor may be operable to determine at least one parameter of the increase in hydrogen sulfide concentration. The at least one parameter may be selected from the group consisting of maximum, minimum, mean, median, standard deviation, mode and percentiles. The apparatus may further comprise a GPS locator operably coupled to the processor configured to indicate a location of the apparatus.

The apparatus may further comprise an output display operable to display a result to a user as provided by the processor. The apparatus may further comprise a cover operable to enclose the cavity. The cover may be selectably sealable to the body.

According to a further embodiment, there is disclosed a method of testing geological samples comprising locating a body defining a cavity thereon over a sample to be tested, measuring an initial hydrogen sulfide concentration within the cavity, introducing a quantity of an oxidizing agent to the sample, measuring a second hydrogen sulfide concentration within the cavity and comparing the initial and oxidized hydrogen sulfide concentrations to indicate a potential for the presence of a desired mineral.

The initial measurement is formed of a plurality of measurements. The initial measurement is formed of a continuous averaged measurement. The oxidized measurement is formed of a plurality of measurements. The oxidized measurement is formed of a continuous averaged measurement.

The method may further comprise calculating with a processor operably coupled to a sensor in communication with the cavity at least one parameter of the increase in hydrogen sulfide concentration within the cavity after the introduction of the oxidizing agent. The at least one parameter may be selected from the group consisting of maximum, minimum, mean, median, standard deviation, mode and percentiles.

Other aspects and features of the present disclosure will become apparent to those ordinarily skilled in the art upon review of the following description of specific embodiments in conjunction with the accompanying figures.

### BRIEF DESCRIPTION OF THE DRAWINGS

The accompanying drawings constitute part of the disclosure. Each drawing illustrates exemplary aspects wherein similar characters of reference denote corresponding parts in each view,

FIG. 1 is a perspective view of an apparatus for testing soil and rock samples according to a first embodiment of the present disclosure applied to a rock.

FIG. 2 is a cross sectional view of the apparatus of FIG. 1 as taken along the line 2-2.

FIG. 3 is a flow chart representing a method for testing geological samples using the apparatus of FIG. 1.

FIG. 4 is a graphical illustration of test sample measurements for use in the method of FIG. 3.

FIG. 5 is a cross sectional view of an apparatus for testing soil and rock samples according to a further embodiment of the present disclosure.

FIG. 6 is a schematic of the apparatus of FIG. 1.

### DETAILED DESCRIPTION

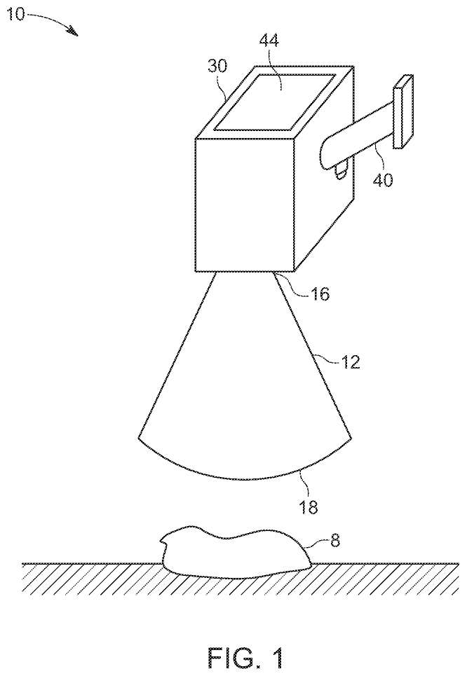

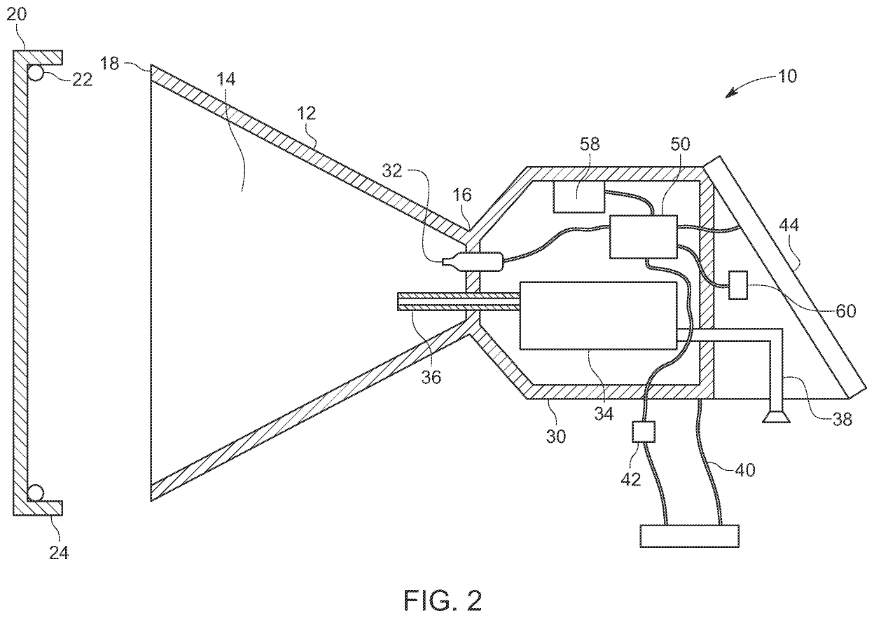

Aspects of the present disclosure are now described with reference to exemplary apparatuses, methods and systems. Referring to FIG. 1, an exemplary apparatus for testing geological samples according to a first embodiment is shown generally at 10. The apparatus 10 comprises an isolation body 12 with an associated hydrogen sulphide sensor 32 (illustrated in FIG. 2) and a source for an oxidizing agent.

As illustrated in FIGS. 1 and 2, the apparatus comprises an isolating body 12 and a support body 30. The isolating body 12 defines an interior cavity 14 into which a soil or rock sample may be located. Alternatively, the isolating body 12 may be located over the soil or rock 8 sample which may be too large or in situ to be easily moved. The interior cavity 14 will have a size selected to have such samples located therein while permitting the apparatus 10 to be easily transportable. In particular, it has been found that a size of between 6 and 36 inches in diameter has been useful for individually portable uses, although it will be appreciated that other sizes may also be useful. As illustrated, the isolation body 12 may have a frustoconical shape with the support body at a proximate or tapered end 16 and extending to a distal or base edge 18. Optionally, the isolating body 12 may include a cover 20 operable to cover the bottom end of the cavity 14 with a lip 24 or other known means. A seal 22 of any known design may also be included to seal the connection between the cover 20 and the isolating body 12. It will be appreciated that other shapes may also be useful, including, without limitation, semi-spherical, cubical or irregular.

The tapered end 16 is connected to the support body 30. The support body 30 includes a hydrogen sulphide sensor 32 operably connected to a processor 50 operable to measure at least two concentration of hydrogen sulphide within the cavity. It will be appreciated that the sensor will be selected to measure an expected concentration of hydrogen sulphide to be encountered which may be in the parts per million or parts per billion ranges. It will be appreciated that any suitable sensor type operable to detect hydrogen sulphide will be suitable such as by way of non-limiting example, electrochemical. The support body 30 further includes a reservoir 34 containing a quantity of an oxidizing agent operable to be dispensed therefrom through a conduit 36 into the cavity 14. The reservoir 34 may be selected to be of any type including, without limitation, rigid, flexible or pressurized and may be optionally fillable through a fill port 38 operable to connect to a remote reservoir for refilling or refilling with a hose or other commonly known means. Any suitable oxidizing agent may be provided therein, including, without limitation, any known gas or liquid oxidizing agent such as, by way of non-limiting example, water, oxygen, ozone, hydrogen peroxide, potassium permanganate or halogens.

As illustrated in FIG. 2, the reservoir 34 may include a fill port or passage 38 extending thereto for refilling the reservoir 34. The support body 30 may also include one or more handles 40 a trigger 42 or other activating device and a screen for outputting results to a user indicating measurements from the hydrogen sulphide sensor 32 or interpretations of the results as provided by the processor 50. Optionally, the apparatus 10 may include a GPS locator 58 as are commonly known, operable to indicate to the processor 50, the location of the apparatus and a data output port 60. The data output port 60 may be of any conventional type so as to permit the transfer of data from the processor 50 and memory 52 to an external source. By way of non-limiting example the data port may be selected from wired, such as usb types, Ethernet, hdmi, coaxial or wireless including wifi or Bluetooth by way of non-limiting example.

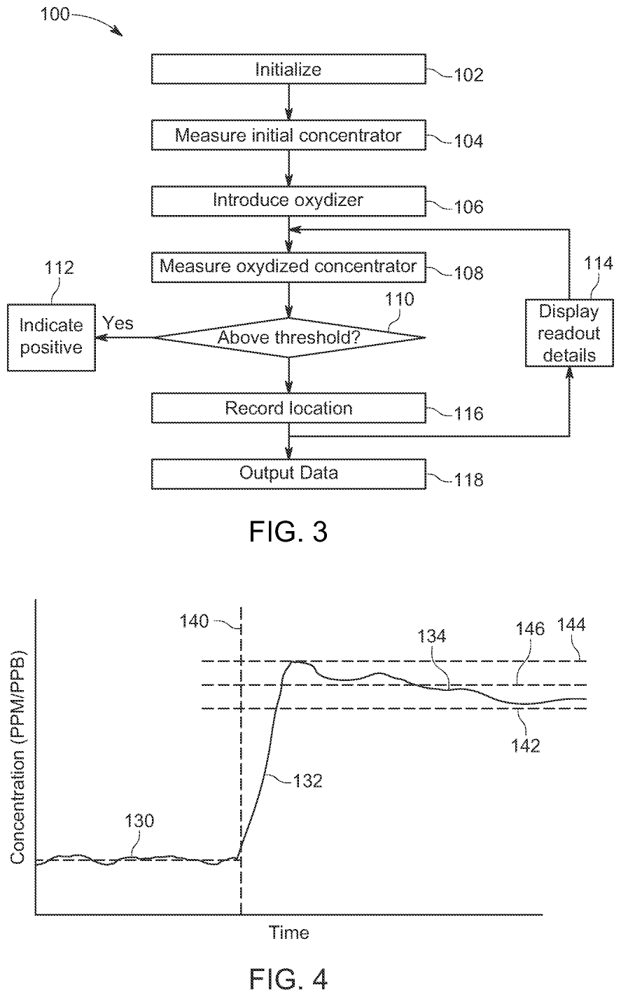

Turning now to FIG. 3 a method for utilizing the apparatus 10 for testing a rock or soil sample is shown generally at 100. The method may optionally comprise initializing or preparing the hydrogen sensor at step 102. Thereafter an initial hydrogen sulphide measurement may be taken at step 104. In operation, the initial measurement may comprise an instantaneous measurement, a continuous measurement over a period of time or a series of averaged measurements. The initial measurement is taken of the sample 8 at a natural or as found state so as to provide a measure of the hydrogen sulphide naturally occurring at that location. Thereafter, the apparatus 10 will introduce or apply a quantity of the oxidizing agent from the reservoir 34 to the sample 8 in step 106 and a second or oxidized measurement measured in step 108. Similar to the initial measurement, the oxidized measurement may comprise an instantaneous measurement, a continuous measurement over a period of time or a series of averaged measurements.

After recording the oxidized measurement, the processor 50 may compare the initial and oxidized measurements so as to determine an increase in hydrogen sulphide concentration after the application of the oxidizing agent in step 110. In particular, the processor 50 may be configured to determine if the increase is above a predetermined amount and indicate a positive indication in step 112 if it is above such predetermined threshold.

Furthermore, the processor 50 may be configured to determine and display in step 114 an output representing one or more parameters of the measured hydrogen sulphide concentration on the display 44 in step 114. In particular, the one or more parameters may include but are not necessarily limited to minimum measurement 142, maximum measurement 144, mean measurement 146 median measurement, standard deviation, mode or percentiles. It will also be appreciated that the processor 50 may be configured to display any or all of these parameters as a change in concentration from before and after the introduction of the oxidizing agent or as a measure of the change in the concentration relative to a measure before oxidization. As illustrated in FIG. 3, the measured oxidized concentration may be repeated and the results updated as many times as required by a user.

Optionally, as illustrated in FIG. 3, the processor 50 may record a location for the data as provided by the GPS locator 58 in step 116. The combined data associated with that sample may then be optionally uploaded or otherwise transmitted in step 118 through the output 60. It will be appreciated that the inclusion of location of the particular sample 8 with the measurements of initial and oxidized concentrations will provide increased quality control of the measurements by ensuring the proper samples for a desired location are utilized. The location of each sample may also be aggregated with other similar measurements to map or plot a plurality of samples to determine geological profiles and areas.

As illustrated in FIG. 4, the initial and oxidized measurements 130 and 134 may be different points in a continuous measurement of hydrogen sulphide. Upon introduction of the oxidizing agent at a time generally indicated at 132, an increase in the hydrogen sulphide may be observed. The processor may be adapted to continuously monitor and interpret such increases as to be operable to extrapolate the quantity of hydrogen sulphide emitted from the sample upon introduction of the oxidizing agent. As illustrated, the maximum 144, minimum 142, mean 146 and other measurements after the introduction of the oxidizing agent at 140 may be determined.

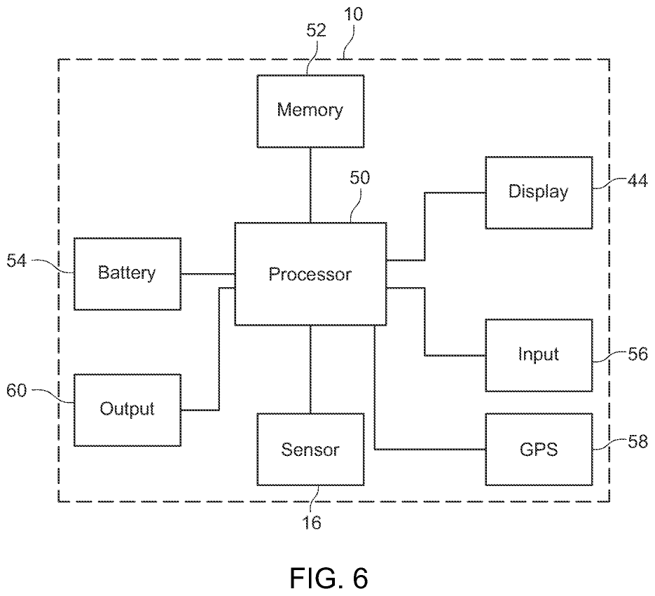

Turning now to FIG. 6, the apparatus 10 includes the processor 50, and memory 52 that stores machine instructions that, when executed by the processor 50, cause the processor 50 to perform one or more of the operations and methods described herein. The memory 52 may be of any known type including a cache memory unit for temporary local storage of instructions, data, or computer addresses as well as storing any data collected by the apparatus 10. The apparatus may optionally include the output display 44, an input 56 for inputting instructions or data to the memory 52 and a batter 54. As outlined above, the processor 50 is adapted to interface with the hydrogen sulphide sensor 32 and optionally the memory 52 to measure, compare and interpret the initial and oxidized measurements 130 and 134 so as to display a result or interpretation thereof to a user.

More generally, in this specification, the term “processor” is intended to broadly encompass any type of device or combination of devices capable of performing the functions described herein, including (without limitation) other types of microprocessors, microcontrollers, other integrated circuits, other types of circuits or combinations of circuits, logic gates or gate arrays, or programmable devices of any sort, for example, either alone or in combination with other such devices located at the same location or remotely from each other. Additional types of processors will be apparent to those ordinarily skilled in the art upon review of this specification, and substitution of any such other types of processors is considered not to depart from the scope of the present invention as defined herein. In various embodiments, the processor 50 can be implemented as a single-chip, multiple chips and/or other electrical components including one or more integrated circuits and printed circuit boards.

Computer code comprising instructions for the processor to carry out the various embodiments, aspects, features, etc. of the present disclosure may reside in the memory 52. The code may be broken into separate routines, products, etc. to carry forth specific steps disclosed herein. In various embodiments, the processor 50 can be implemented as a single-chip, multiple chips and/or other electrical components including one or more integrated circuits and printed circuit boards. The processor 50 together with a suitable operating system may operate to execute instructions in the form of computer code and produce and use data. By way of example and not by way of limitation, the operating system may be Windows-based, Mac-based, or Unix or Linux-based, among other suitable operating systems. Operating systems are generally well known and will not be described in further detail here.

Memory 52 may include various tangible, non-transitory computer-readable media including Read-Only Memory (ROM) and/or Random-Access Memory (RAM). As is well known in the art, ROM acts to transfer data and instructions uni-directionally to the processor 50, and RAM is used typically to transfer data and instructions in a bi-directional manner. In the various embodiments disclosed herein, RAM includes computer program instructions that when executed by the processor 50 cause the processor 50 to execute the program instructions described in greater detail below. More generally, the term “memory” as used herein encompasses one or more storage mediums and generally provides a place to store computer code (e.g., software and/or firmware) and data. It may comprise, for example, electronic, optical, magnetic, or any other storage or transmission device capable of providing the processor 50 with program instructions. Memory 52 may further include a floppy disk, CD-ROM, DVD, magnetic disk, memory chip, ASIC, FPGA, EEPROM, EPROM, flash memory, optical media, or any other suitable memory from which processor 50 can read instructions in computer programming languages.

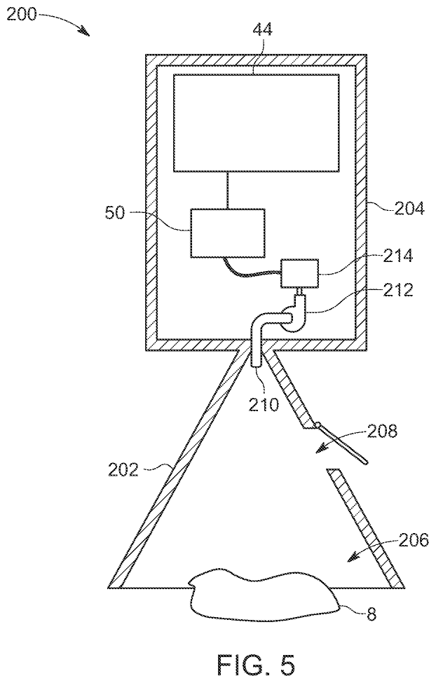

Turning now to FIG. 5, an alternative embodiment of the apparatus for testing geological samples according to a first embodiment is shown is illustrated at 200. The apparatus 200 comprises an isolation body 202 and a support body 204. The isolation body forms a cavity 206 into which a sample 8 may be located and may include a port 208 therethrough for the introduction of an oxidizing agent. The apparatus 200 may include a passage 210 extending away from the cavity 206 extending to a pump or vacuum 212 in communication with a hydrogen sulphide sensor 214 so as to draw air from the cavity and provide it to the sensor. The sensor 214 is in communication with the processor 50 as set out above.

While specific embodiments have been described and illustrated, such embodiments should be considered illustrative only and not as limiting the disclosure as construed in accordance with the accompanying claims.

What is claimed is:

1. An apparatus for testing geological samples comprising:
a body sized to be located over a testing location defining a cavity therein;
a reservoir containing an oxidizing agent;
an applicator configured to dispense a quantity of the oxidising agent from the reservoir to a sample located with or under the cavity; and
a sensor adapted to measure a concentration of hydrogen sulphide within the cavity.

2. The apparatus of claim 1 wherein the body is substantially frustoconical.

3. The apparatus of claim 1 further comprising a processor operable to record the initial and oxidized measurements as provided by the sensor and determine an increase in hydrogen sulfide concentration in response to the introduction of the oxidizing agent.

4. The apparatus of claim 3 wherein the processor is operable to determine at least one parameter of the increase in hydrogen sulfide concentration.

5. The apparatus of claim 4 wherein the at least one parameter is selected from the group consisting of maximum, minimum, mean, median, standard deviation, mode and percentiles.

6. The apparatus of claim 4 further comprising a GPS locator operably coupled to the processor configured to indicate a location of the apparatus.

7. The apparatus of claim 3 further comprising an output display operable to display a result to a user as provided by the processor.

8. The apparatus of claim 1 further comprising a cover operable to enclose the cavity.

9. The apparatus of claim 8 wherein the cover is selectably sealable to the body.

10. A method of testing geological samples comprising:
locating a body defining a cavity therein over a sample to be tested;
measuring an initial hydrogen sulfide concentration within the cavity;
introducing a quantity of an oxidizing agent to the sample;
measuring a second hydrogen sulfide concentration within the cavity; and
comparing the initial and oxidized hydrogen sulfide concentrations to indicate a potential for the presence of a desired mineral.

11. The method of claim 10 wherein the initial measurement is formed of a plurality of measurements.

12. The method of claim 10 wherein the initial measurement is formed of a continuous averaged measurement.

13. The method of claim 10 wherein the oxidized measurement is formed of a plurality of measurements.

14. The method of claim 10 wherein the oxidized measurement is formed of a continuous averaged measurement.

15. The method of claim 10 further comprising calculating with a processor operably coupled to a sensor in communication with the cavity at least one parameter of the increase in hydrogen sulfide concentration within the cavity after the introduction of the oxidizing agent.

16. The method of claim 15 wherein the at least one parameter is selected from the group consisting of maximum, minimum, mean, median, standard deviation, mode and percentiles.
